(19) United States
(12) Patent Application Publication
(10) Pub. No.: US 2017/0315130 A1
(43) Pub. Date: Nov. 2, 2017

(54) EARLY PREDICTION OF PREECLAMPSIA

(71) Applicant: UNIVERSITY OF IOWA RESEARCH FOUNDATION, Iowa City, IA (US)

(72) Inventors: Justin L. Grobe, Iowa City, IA (US); Mark K. Santillan, Iowa City, IA (US); Donna Ann Santillan, Iowa City, IA (US)

(21) Appl. No.: 15/583,400

(22) Filed: May 1, 2017

Related U.S. Application Data
(60) Provisional application No. 62/329,829, filed on Apr. 29, 2016.

(51) Int. Cl. G01N 33/68; C12Q 1/68

(52) U.S. Cl. CPC G01N 33/689; C12Q 1/6883; G01N 2800/368; C12Q 2600/158

(57) ABSTRACT

The invention relates to methods and kits for diagnosing or predicting the likelihood of occurrence of preeclampsia in a subject with no history of the disease.

### CROSS-REFERENCE TO RELATED APPLICATIONS

This application claims the benefit of U.S. Provisional Patent Application No. 62/329,829, filed Apr. 29, 2016, which is incorporated by reference herein in its entirety.

### BACKGROUND OF THE INVENTION

### Field of the Invention

This disclosure relates to improving the early prediction of preeclampsia in women with no history of the disorder.

### Description of Related Art

Preeclampsia is a serious hypertensive disorder in pregnancy that can cause maternal complications including headaches, edema, liver and renal damage, seizures, and death. Women who experience preeclampsia during pregnancy are also at a greater life-long risk for cardiovascular diseases including hypertension, stroke, myocardial infarction, and cardiovascular death. According to the 2011 California Pregnancy Associated Mortality Review, a delay in the diagnosis of preeclampsia contributed to the cause of 92% of the maternal deaths in California among women with preeclampsia (see The California Pregnancy-Associated Mortality Review. Report from 2002 and 2003 Maternal Death Reviews. Sacramento, Calif.: California Department of Public Health, Maternal Child and Adolescent Health Division; 2011). Although an early predictor of preeclampsia could potentially save lives, there is no simple or reliable method currently available in clinical use to predict which women early in pregnancy will eventually develop preeclampsia.

Recently, it has been established that elevated maternal plasma copeptin, the pro-segment of arginine vasopressin, is highly predictive of the development of preeclampsia (see PCT/US2014/015627, PCT/US2014/015631, and Santillan M K, Santillan D A, Scroggins S M, Min J Y, Sandgren J A, Pearson N A, Leslie K K, Hunter S K, Zamba G K, Gibson-Corley K N, Grobe J L. Vasopressin in preeclampsia: A novel very early human pregnancy biomarker and clinically relevant mouse model. Hypertension, 2014). It was shown that preeclampsia could be predicted as early as the 6th week of gestation despite that clinical symptoms do not typically occur until after the 20th-24th week of gestation with most cases developing in the late third trimester. The case-controlled study demonstrated that copeptin is robustly predictive of the development of preeclampsia in the 1st, 2nd, and 3rd trimester. Furthermore, clinically significant sensitivity, specificity, negative predictive value, and positive predictive value were demonstrated for copeptin even when controlling for significant confounders (Santillan et al.).

However, because women in their first pregnancy are likely not to be as familiar with the signs and symptoms of preeclampsia, they are more likely to attribute headache, edema, and right upper quadrant pain to being a normal part of pregnancy. In contrast, women with a history of preeclampsia are at higher risk for a second pregnancy complicated by preeclampsia and are more aware of the signs and symptoms of the disease. Importantly, prenatal care providers are more diligent in watching for the development of preeclampsia in a patient population with a preexisting history than women who are in their first pregnancy. Moreover, it is clinically useful to distinguish women with a history of preeclampsia from women with no history of the disease. Therefore, there is a need for a way to determine accurately the risk for preeclampsia in women with no history of the disease.

### SUMMARY OF THE INVENTION

It is against the above background that the present invention provides certain advantages and advancements over the prior art. In particular, as set forth herein, the use of copeptin to predict the onset of preeclampsia is disclosed.

Although this invention disclosed herein is not limited to specific advantages or functionalities, in a first aspect, the invention provides a method of predicting the occurrence of preeclampsia in a pregnant woman, the method including the steps of collecting a bodily sample from a subject during the first trimester of pregnancy, applying the bodily sample to an assay adapted to detect copeptin in the bodily sample, measuring a copeptin concentration in the bodily sample, and predicting the occurrence of preeclampsia in the pregnant woman. The assay provides greater sensitivity for predicting the occurrence of preeclampsia in pregnant women with no history of the disease than in pregnant women with a history of the disease.

In one embodiment of the first aspect, the bodily sample comprises at least one of whole blood, serum, plasma, urine, tissue, cells, sweat, and tears. In one embodiment of the first aspect, the bodily sample can be a fresh sample or a frozen sample. In another embodiment of the first aspect, the step of applying the bodily sample to the assay includes: a) combining the bodily sample with an assay solution comprising a copeptin-specific binding agent to form an assay mixture comprising copeptin-binding agent complexes; and b) applying the assay mixture to a substrate. The measuring step can include measuring the concentration and/or number of copeptin-binding agent complexes. The step of applying the bodily sample to the assay can include: a) applying the bodily sample to a substrate; and b) applying an assay solution to the substrate comprising a copeptin-specific binding agent to form an assay mixture. The copeptin-binding agent can be at least one of an antibody or an antibody fragment. The antibody or antibody fragment can be linked to at least one of an enzyme, a nucleic acid tag, a prosthetic group, a fluorescent material, a luminescent material, a bioluminescent material, a radioactive material, a positron emitting metal, and a nonradioactive paramagnetic metal ion. The antibody or antibody fragment can be chimerized, humanized, or deimmunized.

In a second aspect, the invention provides a method of detecting copeptin in a patient, the method including the steps of obtaining a bodily sample from a patient, applying the bodily sample to an assay adapted to detect copeptin in the bodily sample, and detecting a level of copeptin in the bodily sample. The patient is a pregnant woman with no history of preeclampsia.

In one embodiment of the second aspect, the bodily sample comprises whole blood, serum, plasma, urine, tissue, cells, sweat, or tears. In another embodiment of the second aspect, the bodily sample is a fresh sample or a frozen sample. In another embodiment of the second aspect, the step of applying the bodily sample to the assay comprises: a) combining the bodily sample with an assay solution comprising a copeptin-specific binding agent to form an assay mixture comprising copeptin bound with the copeptin-specific binding agent and applying the assay mixture to a substrate; b) applying the bodily sample to a substrate and applying an assay solution to the substrate comprising a copeptin-specific binding agent to form an assay mixture; or c) applying the bodily sample to a copeptin-specific binding agent bound to a substrate. In one embodiment of the second aspect, the copeptin-specific binding agent comprises an antibody or an antibody fragment. In one embodiment of the second aspect, the antibody or antibody fragment is linked to at least one of an enzyme, a nucleic acid tag, a prosthetic group, a fluorescent material, a luminescent material, a bioluminescent material, a radioactive material, a positron emitting metal, and a nonradioactive paramagnetic metal ion. In one embodiment of the second aspect, the antibody is an IgG₁, IgG₂, IgG₃, IgG₄, IgA₁, IgA₂, IgM, IgE, or IgD antibody, and the antibody fragment is a Fab, a F(ab′)₂, a monospecific Fab₂, a bispecific Fab₂, a trispecific Fab₃, a monovalent IgG, an scFv, a bispecific diabody, a trispecific triabody, an scFv-sc, a minibody, an IgNAR, a V-NAR, an hcIgG, or a VhH. In one embodiment of the second aspect, the bodily sample is obtained during the first trimester of pregnancy.

In a third aspect, a method for identifying a treatment modality for a pregnant subject including obtaining a bodily sample from the subject, wherein the sample is taken during the first trimester of the pregnancy, measuring copeptin levels in the bodily sample using an assay, and identifying a treatment modality for the pregnant subject based on an increased level of copeptin in the bodily sample as measured by the assay. The assay provides greater sensitivity for predicting the occurrence of preeclampsia in pregnant women with no history of the disease than in pregnant women with a history of the disease.

In one embodiment of the third aspect, the increased level of copeptin is predictive of the pregnant subject developing preeclampsia. In one embodiment of the third aspect, the treatment modality is for the treatment of preeclampsia. In one embodiment of the third aspect, the treatment includes administration of at least one of an antihypertensive, a corticosteroid, an anticonvulsant, and a vaptan. In one embodiment of the third aspect, the method enables an earlier initiation of treatment of preeclampsia during the pregnancy of the pregnant subject.

In a fourth aspect, a kit for predicting the occurrence of preeclampsia in a subject includes a binding agent adapted to bind copeptin in a bodily sample taken from the subject during the first trimester of pregnancy. The kit provides greater sensitivity for predicting the occurrence of preeclampsia in pregnant women with no history of the disease than in pregnant women with a history of the disease.

In one embodiment of the fourth aspect, the binding agent comprises at least one of an antibody or an antibody fragment. In one embodiment of the fourth aspect, the antibody or antibody fragment is linked to at least one of an enzyme, a nucleic acid tag, a prosthetic group, a fluorescent material, a luminescent material, a bioluminescent material, a radioactive material, a positron emitting metal, a nonradioactive paramagnetic metal ion. In one embodiment of the fourth aspect, the antibody is an IgG₁, IgG₂, IgG₃, IgG₄, IgA₁, IgA₂, IgM, IgE, or IgD antibody, and the antibody fragment is a Fab, a F(ab′)₂, a monospecific Fab₂, a bispecific Fab₂, a trispecific Fab₃, a monovalent IgG, an scFv, a bispecific diabody, a trispecific triabody, an scFv-sc, a minibody, an IgNAR, a V-NAR, an hcIgG, or a VhH.

In one embodiment of the fourth aspect, the kit enables quantification of copeptin in the bodily sample by quantitative PCR, epitope pull down via antibody-linked magnetic particles, column chromatography, gas chromatography, mass spectrometry, fluorescence, color change, flow cytometry, tissue staining, densitometry, western blot, or bio-barcode. In one embodiment of the fourth aspect, the kit can further include one or more binding agents adapted to bind one or more of cell-free fetal DNA, cell-free total DNA, and pregnancy-associated plasma protein A. In one embodiment of the fourth aspect, the kit can include at least one binding agent adapted to detect aneuploidy, alpha-fetoprotein, or human chorionic gonadotropin.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings in which:

FIG. 1 shows a receiver operator curve analysis for a cohort with no history of preeclampsia; and

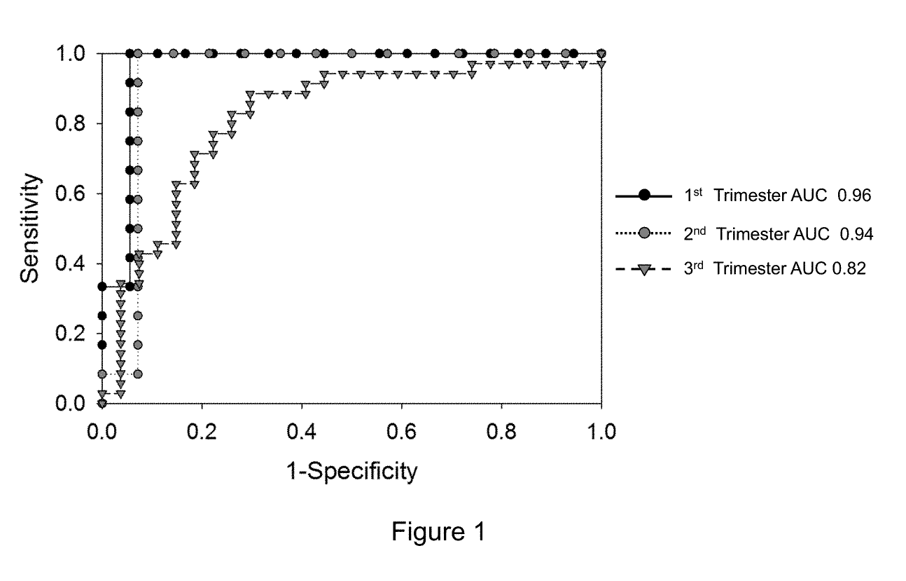

FIG. 2 shows that plasma copeptin levels in women with no history of preeclampsia who later develop preeclampsia have significantly elevated copeptin throughout gestation (*=P<0.05).

### DETAILED DESCRIPTION OF THE INVENTION

All publications, patents, and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to an “antibody” means one or more antibodies.

It is noted that terms like “preferably,” “commonly,” and “typically” when used herein are not utilized to limit the scope of the description or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “about” indicates ±10% of a given value.

As used herein, the term “bodily sample” or “patient sample” or “experimental sample,” or “sample” interchangeably refer to whole blood, blood fractions, including separately serum and/or plasma, urine, tissue, a biopsy, cells, and bodily fluids, including, for example, sweat and tears, and any combination thereof isolated from an individual. Such samples may be fresh, frozen, or otherwise stored.

The present invention is based, at least in part, on the discovery that early measurement of copeptin levels during pregnancy is predictive of a subject developing preeclampsia later in pregnancy. However, more particularly, the present invention is based on the new observation that the sensitivity and specificity to predict preeclampsia using copeptin was significantly improved in patients with no history of preeclampsia in comparison to a parent cohort of all patients including women with a history of preeclampsia.

It is contemplated herein that assays and methods for detection of copeptin as described herein may be combined and further coupled with additional assays for preeclampsia including Doppler velocimetry measurements on at least one of a subject's uterine and/or umbilical arteries or other pertinent vasculature including but not-limited to the middle cerebral artery and ductus venosus, as well as primary placental vessel flow measurements using other technologies such as CT or MRI. It is further contemplated that additional assays may be combined with those disclosed herein, such as serum screening for aneuploidy, neural tube defects, and others known in the art. In this way, a single device can be used to screen for multiple conditions that may affect the mother and/or the fetus.

Contemplated methods and kits for diagnosing or predicting the likelihood of occurrence of preeclampsia in a subject with no history of preeclampsia may include one or more antibody detection or other assays (test assays) specific for at least the detection of copeptin in a sample taken from the subject. The sample is taken early in pregnancy from the subject, for example, in the first trimester of pregnancy. While antibody-based detection assays are contemplated herein, additional test assays or detection assays such as copeptin-specific assays or other specific assays that are specific for the protein products of the vasopressin gene are also contemplated herein, including, for example, protein- and/or peptide-specific assays, enzyme activity assays (enzyme detection assays), immune-PCR-based techniques employing nucleic acid-linked antibodies that can be measured by quantitative PCR, epitope pull down via antibody-linked magnetic particles, including nanoparticles, or other selectable tag, mass spectrometry, and combinations thereof. Kits contemplated herein may include positive and negative control samples, assay reagents, as well as instructions.

Samples contemplated in the present disclosure include whole blood, blood fractions, including serum and/or plasma, urine, tissues, cells, and bodily fluids, including, for example, sweat and tears, and any combination thereof. One preferred sample is plasma. Another preferred sample is serum. Another preferred sample is urine. In one embodiment, a kit includes an antibody detection assay that can be used with plasma, serum and/or urine, in other words, any bodily sample may be used for the single assay.

A contemplated assay may include a test strip, an ELISA, or other antibody-based or other target-specific assay, such as an enzyme activity assay where the presence of a targeted enzyme is detected by chromogenic means and the like due to enzyme activity. Test strips may be prepared in the conventional manner such as is described in U.S. Pat. No. 6,210,971 or 5,733,787 to Bayer Corporation (Elkhart, Ind.). It is contemplated that the test strips may couple attachment of the targeted epitope with the initiation of one or more of a chromogenic, fluorogenic, or luminescent reaction, as is known in the art, to indicate binding of the desired target. Further, a test strip can be characterized as an absorbent substrate capable of immobilizing metabolites bound to a layer of support material. Well-known solid phase supports may include paper, cellulose, fabrics made of synthetic resin, e.g. nylon or unwoven fabric. The absorbent material is typically bound to a layer of support material such as glass fiber or a synthetic polymer sheet to provide structural support. Other suitable solid phase supports are contemplated herein.

Additional assay formats contemplated for use include dipsticks (allowing dipping of the assay device into a test sample), urine tests (configured to allow an individual to urinate onto an assay device), finger prick with test strip or disk formats (similar to blood glucose and/or cholesterol assays), and other technologies. In one embodiment, assay formats may be designed for single use, at home testing by an individual. In another embodiment, assay formats may be multiplexed for replication within a testing format, such as a testing format that include 2 or more tests for repeat testing at the same time and averaging of results. In a further embodiment, contemplated assay formats may be multiplexed for testing samples from multiple individuals at the same time, such as, for example only, in a 96-well plate format, where up to 96 different samples may be tested at the same time. Different numbers of tests (i.e., repeats of the same test) are contemplated for each assay format.

In another embodiment, contemplated diagnostic platforms include measurement of copeptin levels from a bodily sample using flow cytometry, fluorescence, color change, tissue staining, quantitative PCR, densitometry, western blot, bio-barcode, and the like.

Further, two (or more, such as three or four) assays may be combined in a single assay device, such as, for example a pregnancy test that uses chromogenic or other means (for example, based on urine analysis or other sample). In this embodiment, in addition to the pregnancy test, one or more tests for prediction of preeclampsia would be included. In this embodiment, a “positive” result for pregnancy (the subject is pregnant) may be indicated by a first indicium and a “positive” result for the preeclampsia test (indicating a predisposition for preeclampsia) may be indicated by second indicium.

In another embodiment, a three test assay is contemplated that tests for pregnancy and multiple preeclampsia predictive markers, such as copeptin and LNPEP. In this way, a greater specificity for prediction of preeclampsia accompanying pregnancy may be had in a single test.

Test assays may be incorporated into single use devices that may be purchased by the end user (for example, a woman seeking to know whether she is pregnant and at risk for preeclampsia). The test assay devices may be employed by application of a urine, blood, and other some other sample to a single or multiple portions thereof, incubating the test assay for a prescribed period of time, such as about 1 minute, about 5 minutes, about 10 minutes, about 15 minutes, or about 1 hour, and comparing the result to an interpretation key associated with a package in which the test assay device was purchased or on the test assay device itself.

Copeptin levels in a sample from a pregnant woman with no history of preeclampsia compared to control are predictive of the occurrence of preeclampsia in the woman at levels of at least about 500 pg/mL, or at least 550 pg/mL, or at least 600 pg/mL, or at least 700 pg/mL, or at a fold increase of at least about 1/100 fold, or about 1/50 fold, or about 1/25 fold, or about 1/16 fold, or about ⅛ fold, or about ¼ fold, or about 2 fold, or greater or less.

Similarly, decreases in LNPEP levels in a sample compared to control are considered to be predictive of the occurrence of preeclampsia during the subject's pregnancy, including, for example, of about 1/100 fold, or about 1/50 fold, or about 1/25 fold, or about 1/16 fold, or about ⅛ fold, or about ¼ fold, or about 2 fold, or greater or less.

In one embodiment, a method of diagnosing or predicting the likelihood of occurrence of preeclampsia in a subject with no history thereof may include collecting a sample, such as, urine, from the subject during the first trimester of pregnancy, measuring copeptin levels in the sample using, for example, an antibody detection assay or other assay, and determining whether the subject is likely to develop preeclampsia later in pregnancy by comparing the subject's copeptin levels to a control. Assays may provide data, for example, by color changes, light emission, changes in light emission intensity, densitometry, or changes in opacity/translucence of a substrate. These data, in turn, may be converted to data points that may be plotted compared to controls.

All methods considered herein can further be combined with another diagnostic assay, such as Doppler velocimetry measurements on at least one of the subject's uterine and umbilical arteries or other pertinent vasculature including but not limited to the middle cerebral artery and ductus venosus, as well as primary placental vessel flow measurements using other technologies such as CT or MRI.

By early pregnancy, we mean at least before 20 weeks of amenorrhea, more preferably, at least before about 16, or about 12, or about 8, or about 6 weeks, or about 4 weeks of pregnancy. Early in pregnancy may also be during the first trimester.

By “patient” or “subject,” it is meant a female subject, such as, a human. Controls contemplated herein may comprise a single healthy pregnant age-matched subject, or a population of multiple healthy pregnant age-matched subject subjects or multiple healthy pregnant subjects, or serum and/or urine samples from a population of multiple healthy pregnant subjects none of whom later develop preeclampsia during pregnancy. Controls may further include a partially or fully purified copeptin standard that is included in an assay in parallel with a patient sample for comparison. In addition, a predetermined control may also be a negative predetermined control. For example, a negative predetermined control comprises one or multiple subjects who developed preeclampsia during pregnancy. It is further contemplated that LNPEP and/or copeptin levels in a patient sample may be normalized to a total protein value of the sample for analysis.

Antibody detection assays contemplated here may include assays that use antibodies or antibody fragments to target a specific molecule of interest. Detection of the molecule may occur via antibody attachment to the molecule in combination with an indicator associated with the antibody or antibody fragment. It is further envisioned that the molecule of interest, for example copeptin or other AVP gene protein product, may be measured by column chromatography, gas chromatography, mass spectrometry, and combinations thereof. Examples of indicators to be attached to antibodies contemplated herein include various enzymes, a nucleic acid tag that can be used in immuno-PCR, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions, which can be conjugated to antibodies for use as diagnostics according to the present invention. Non-limiting examples of suitable enzymes include horseradish peroxidase, alkaline phosphatase, betagalactosidase, or acetylcholinesterase; non-limiting examples of suitable prosthetic group complexes include streptavidin/biotin and avidin/biotin; non-limiting examples of suitable fluorescent materials include umbelliferone, fluorescein, fluorescein isothiocyanate, rhodamine, dichlorotriazinylamine fluorescein, dansyl chloride or phycoerythrin; a non-limiting example of a luminescent material includes luminol; non-limiting examples of bioluminescent materials include luciferase, luciferin, and aequorin; and non-limiting examples of suitable radioactive material include 125I, 131I, 111In, or 99Tc.

One example of an antibody detection assay is an ELISA. An ELISA may include antibodies or antibody fragments specific for antigens or epitopes of copeptin or other coexpressed regions of the protein product of the vasopressin (AVP) gene, such as vasopressin and neurophysin II. An antigen can be a natural or synthetic protein or fragment thereof, polysaccharide, or nucleic acid. Skilled artisans know that antigens can induce an immune response and elicit antibody formation.

Antibodies can be molecules synthesized in response to the presence of a foreign substance, wherein each antibody has specific affinity for the foreign material that stimulated its synthesis. The specific affinity of an antibody need not be for the entire molecular antigen, but for a particular site on it called the epitope (Kindt et al., Kuby Immunology, 6th Edition 574 pps, (2006)).

Antibodies can be, for example, a natural or synthetic protein or fragment thereof or nucleic acids (e.g., aptamers) with protein-binding or other antigen-binding characteristics. Antibodies can be produced in response to antigenic stimuli including, but not limited to, exposure to foreign proteins, microorganisms, and toxins. One of ordinary skill in the art can assess antigen-antibody immunocomplex formation by techniques commonly used in the art. Examples of suitable additional assays to assess immunocomplex formation contemplated herein include phage immunoblot and radioimmunoassay. See, e.g., (Dubovsky et al., J. Immunother. 30:675-683 (2007).

In one embodiment, a contemplated antibody is any of isotypes IgG₁, IgG₂, IgG₃, IgG₄, IgA₁, IgA₂, IgM, IgE, or IgD. In another embodiment, the antibody is an IgG₁ or IgG₄ isotype. In one embodiment, an antibody fragment can be any or a combination of the following: Fab, F(ab′)₂, monospecific Fab₂, bispecific Fab₂, trispecific Fab₃, monovalent IgG, scFv, bispecific diabody, trispecific triabody, scFv-sc, a minibody, IgNAR, V-NAR, hcIgG, or VhH. Antibodies or antigen binding fragments thereof contemplated for use herein can be from any species or represent hybrid antibodies combining heavy chains and light chains from different species, and may be specific for any desired epitope. In another embodiment, antibodies contemplated here can have a S228P core-hinge mutation (numbered according to the EU numbering system; or alternatively S241P according to the Kabat system, see, Kabat et al., Sequences of Proteins of Immunological Interest, 4^{th} ed., United States Government Printing Office, 165-492, 1987; see also Silva et al. Jour. Biol. Chem. 290:5462-5469, 2015).

In certain embodiments, anti-copeptin antibodies, antibody fragments, or antigen binding fragments thereof, can be chimerized, humanized, or deimmunized. In one embodiment, an antibody, or antigen binding fragments thereof, of the invention may be chimeric. A chimeric antibody is an antibody in which different portions of the antibody are derived from different animal species, such as antibodies having a variable region derived from a murine monoclonal antibody and a human immunoglobulin constant region. Methods for producing chimeric antibodies, or fragments thereof, are known in the art. See e.g., Morrison, Science 229:1202, 1985; Oi et al., BioTechniques 4:214, 1986; Gillies et al., J. Immunol. Methods 125:191, 1989; U.S. Pat. Nos. 5,807,715; 4,816,567; and 4,816,397. Techniques developed for the production of “chimeric antibodies” (Morrison et al., Proc. Natl. Acad. Sci. 81:851, 1984; Neuberger et al., Nature 312:604, 1984; Takeda et al., Nature 314:452, 1985) may be employed for the synthesis of said molecules. For example, a genetic sequence encoding a binding specificity of a mouse anti-copeptin antibody molecule may be fused together with a sequence from a human antibody molecule of appropriate biological activity. As used herein, a chimeric antibody is a molecule in which different portions are derived from different animal species, such as those having a variable region derived from a murine monoclonal antibody and a human immunoglobulin constant region, e.g., humanized antibodies.

In another embodiment, an antibody, or antigen-binding fragment thereof, of the invention is humanized. Humanized antibodies have a binding specificity comprising one or more complementarity determining regions (CDRs) from a non-human antibody and framework regions from a human antibody molecule. Often, framework residues in the human framework regions will be substituted with the corresponding residue from the CDR donor antibody to alter, preferably improve, antigen binding. These framework substitutions are identified by methods well known in the art, e.g., by modelling of the interactions of the CDR and framework residues to identify framework residues important for antigen binding and sequence comparison to identify unusual framework residues at particular positions. See e.g. Queen et al., U.S. Pat. No. 5,585,089; Riechmann et al., Nature 332:323, 1988. Antibodies can be humanized using a variety of techniques known in the art including, for example, CDR-grafting (EP 239,400; International Publication No. WO 91/09967; U.S. Pat. Nos. 5,225,539; 5,530,101; and 5,585,089), veneering or resurfacing (EP 592,106; EP 519,596; Padlan, Molecular Immunology 28:489, 1991; Studnicka et al., Protein Engineering 7:805, 1994; Roguska. et al., PNAS 91:969, 1994), and chain shuffling (U.S. Pat. No. 5,565,332).

In some embodiments, an assay for measuring copeptin, rather than taking a sample from a patient, includes introducing an anti-copeptin antibody or antibody fragment into the patient and measuring copeptin levels in situ. In such embodiments, de-immunization can be used to decrease the immunogenicity of the antibody, antibody fragment, or antigen binding fragment thereof. As used herein, the term “de-immunization” includes alteration of an antibody, antibody fragment, or antigen binding fragment thereof, to modify T cell epitopes (see, e.g., International Publication Nos. WO9852976A1, WO0034317A2). For example, VH and VL sequences from the starting antibody can be analyzed and a human T cell epitope “map” may be generated from each V region showing the location of epitopes in relation to complementarity-determining regions (CDRs) and other key residues within the sequence. Individual T cell epitopes from the T cell epitope map may be analyzed in order to identify alternative amino acid substitutions with a low risk of altering activity of the final antibody. A range of alternative VH and VL sequences may be designed comprising combinations of amino acid substitutions and these sequences may be subsequently incorporated into a range of copeptin-specific antibodies, antibody fragments, or antigen binding fragments thereof for use in the methods disclosed herein, which are then tested for function. Typically, between 12 and 24 variant antibodies may be generated and tested.

In another embodiment similar to dialysis, it is contemplated to sample blood from a patient by passing the patient's blood through a column or functionally similar device that captures copeptin or vasopressin and allows the blood to return to the patient. In this embodiment, it is contemplated that elevated copeptin levels can be measured and when such levels indicate the need, the process can be used to reduce copeptin and/or vasopressin from the patient's blood stream. A similar system can be used to test waste samples from a patient for elevated copeptin levels, for example, where a urine sample is passed through a column or similar device packed with a medium to which are attached anti-copeptin antibodies or similar copeptin-specific binding agent.

In a further embodiment, contemplated tests for early prediction of preeclampsia can combine measurement of copeptin levels with one or more of cell-free fetal DNA, cell-free total DNA, and pregnancy-associated plasma protein A levels.

In one embodiment, methods of predicting preeclampsia in a pregnant woman can include combining a bodily sample from the woman with an assay solution. The assay solution can include buffers, saline, antigen-binding agents (e.g., antibodies, antibody fragments, or antigen binding fragments thereof specific for copeptin each alone or in combination and other antigen specific binding agents), nucleotides, salts, nucleic acid primers, DNA polymerases, fluorescent compounds, and combinations thereof, which enable quantification of antigen or protein found within the sample. In some embodiments, a copeptin-specific binding agent is included in the assay solution, which is adapted to complex with copeptin in the solution to form an assay mixture comprising copeptin-binding agent complexes. The assay mixture can be assayed subsequently to measure the amount of copeptin and/or number of copeptin-binding agents. As an alternative, the bodily sample can be first applied to a substrate of an assay and an assay solution can be subsequently added to the bodily sample.

### Examples

The Examples that follow are illustrative of specific embodiments of the invention, and various uses thereof. They are set forth for explanatory purposes only, and are not to be taken as limiting the invention.

### Example No. 1. Nested Case Control Study Design of Women with No History of Preeclampsia

### Overview

Preeclampsia annually kills 76,000 mothers and 500,000 babies worldwide often due to delay in diagnosis secondary to the lack of simple, early gestation tests. Elevated circulating copeptin (CPP), the pro-segment of vasopressin, is associated with preeclampsia (PreE). We have previously demonstrated that CPP is robustly predictive of PreE as early as the 6th week of gestation in all mothers. Development of PreE is increased 3-fold by a history of PreE. Therefore, women with a history of preeclampsia are watched more closely for development of preeclampsia than women with no history of the disease. Currently, no test robustly predicts PreE in women without a history of PreE. Such a test could considerably reduce the delay in medical intervention for women with no history of the disease.

To evaluate if CPP is predictive in a low risk setting where a predictor is most needed, a nested case-control study was performed to evaluate the predictive characteristics of CPP of women with and without a history of PreE. Maternal plasma CPP concentrations throughout gestation were measured by ELISA.

### Materials and Methods:

The present study was a nested case control study design of women with no history of preeclampsia. Bivariate comparisons were performed and receiver operating characteristic (ROC) curves were constructed to determine sensitivity, specificity, positive and negative predictive values for particular cutoffs. Multivariable logistic regression was performed to control for confounding variables to examine if CPP was significantly predictive of PreE.

Clinical data were extracted from the electronic medical record (EPIC). Clinical data extracted included maternal race, BMI at the new OB visit; whether the patient was diabetic and if so, what type (type I, II, or gestational), whether or not the patient was a chronic hypertensive, history of preeclampsia and type, whether the patient was preeclamptic in current pregnancy and type, smoking status, maternal age, gravida and parity, gestational age at delivery, birth weight, and APGAR scores.

Sample collection: Maternal blood collected in ACD-A tubes (Becton Dickinson) by the Maternal Fetal Tissue Bank (IRB#200910784) at the University of Iowa Hospitals & Clinics. Plasma was isolated, aliquoted, snap frozen, and stored at −80° C.

Copeptin assay: Copeptin was measured in plasma by colorimetric enzyme-linked immunosorbent assay (ELISA) per the manufacturer's protocol (USCN, Houston, Tex.). Statistical Analysis: Sensitivity, specificity, negative predictive value, and positive predictive value were determined.

Continuous variables: the Student's t-test or ANOVA was utilized. For non-parametric testing: Mann-Whitney test or ANOVA on Ranks was utilized. Categorical variables: Chi square or Fisher exact test was utilized. Logistic regression models were constructed using regression identified and clinically significant confounding variables. Receiver operating characteristic curves were constructed to determine sensitivity, specificity, negative predictive value, and positive predictive value. All variables were tested at significance level of P<0.05.

### Results:

Apart from a difference in prior history of PreE, no significant demographic or clinical differences were observed between groups (see Table Nos. 1 and 2).

TABLE NO. 1
Parent Cohort subject characteristics.

| | Non-pregnant | Control | Preeclampsia | P Value for |
|---|---|---|---|---|
| Characteristics | (n = 33) | (n = 54) | (n = 50) | All Groups |
| Maternal | 31.4 ± 7.2 | 29.9 ± 5.2 | 30.0 ± 5.6 | 0.47 |
| Gravida | 1.0 | 2.0 | 2.0 | <0.001 |
| BM | 29.6 ± 8.5 | 30.0 ± 8.7 | 31.9 ± 9.2 | 0.41 |
| CHTN | 9.1% | 25.9% | 20.0% | 0.16 |
| DM | 3.0% | 20.4% | 22.0% | 0.05 |
| Hx PreE | 0.0% | 29.6% | 18.0% | 0.002 |
| Race: White | 90.9% | 92.6% | 90.0% | 0.56 |
| Race: Hispanic | 0% | 3.7% | 4.0% | 0.56 |
| Race: Asian | 6.1% | 1.8% | 0% | 0.56 |
| Race: Black | 3.0% | 1.9% | 4.0% | 0.56 |

CHTN = chronic hypertensive;
DM = Diabetes Mellitus; and
HxPreE = history of Preeclampsia

TABLE NO. 2
Nested Cohort subject characteristics.

| | Control | Preeclampsia | |
|---|---|---|---|
| Characteristics | (n = 38) | (n = 41) | P Value |
| Maternal Age | 29.3 ± 5.0 | 29.7 ± 5.6 | 0.762 |
| Gravida | 2.4 ± 1.7 | 2.3 ± 1.5 | 0.941 |
| BMI | 30.2 ± 9.9 | 29.9 ± 8.1 | 0.870 |
| CHTN | 26.3% | 24.3% | 0.950 |
| DM | 18.4% | 22.0% | 0.912 |
| Twins | 18.4% | 26.8% | 0.534 |
| Race: White | 92.1% | 87.8% | 0.419 |
| Race: Hispanic | 5.3% | 4.9% | 0.419 |
| Race: Asian | 2.6% | 0% | 0.419 |
| Race: Black | 0% | 4.9% | 0.419 |

In all trimesters, CPP predicted PreE similarly or better in women with no history of PreE as evidenced by an elevated ROC Area Under the Curve in comparison to values of women with a history of Pre (1st trimester: 0.96 vs. 0.90; 2nd trimester: 0.93 vs. 0.90; 3rd trimester: 0.82 vs. 0.78)(see Table No. 3).

TABLE NO. 3
Tested Characteristics of Copeptin.

| Trimester | Parent Cohort | No History of Preeclampsia |
|---|---|---|
| First | Area Under the Curve = 0.90 | Area Under the Curve = 0.96 |
| | Cutoff = 811 | Cutoff = 712 |
| | Sensitivity = 88% | Sensitivity = 92% |
| | Specificity = 81% | Specificity = 94% |
| | P value < 0.001 | P value < 0.001 |
| Second | Area Under the Curve = 0.90 | Area Under the Curve = 0.93 |
| | Cutoff = 866 | Cutoff = 693 |
| | Sensitivity = 81% | Sensitivity = 92% |
| | Specificity = 84% | Specificity = 86% |
| | P value < 0.001 | P value = 0.002 |
| Third | Area Under the Curve = 0.78 | Area Under the Curve = 0.82 |
| | Cutoff = 758 | Cutoff = 783 |
| | Sensitivity = 78% | Sensitivity = 71% |
| | Specificity = 71% | Specificity = 74% |
| | P value < 0.001 | P value < 0.001 |

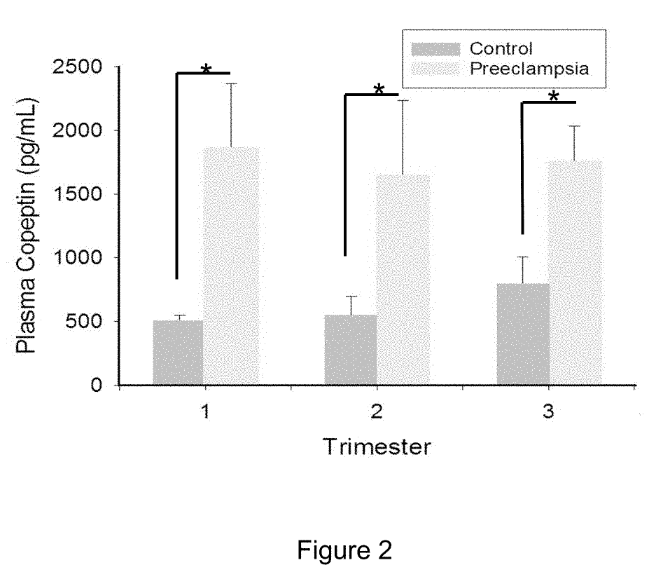

Despite controlling for significant covariates such as maternal age, BMI, diabetes, chronic hypertension, and twin gestation, logistic modeling demonstrate that trimester specific CPP cutoffs throughout gestation were significantly associated with the development of PreE in women with no history of PreE (all models P<0.001)(see FIG. 2). These data indicate that copeptin is more effective as an early predictor of preeclampsia in a low risk cohort than in women with a history of the disease. The ability to predict PreE in such a low risk cohort with CPP is clinically significant because women in whom the diagnosis of preeclampsia is delayed or missed may now receive the appropriate interventions earlier than would have been possible before.

TABLE NO. 4
Logistic Regression Modeling.

| | P Value | P Value significant |
|---|---|---|
| Model | Copeptin | variables |
| First | 0.005 | Hx of PreE = 0.037 |
| Trimester | | |
| Second | 0.009 | Chronic HTN = 0.049 |
| Trimester | | |
| Third | 0.027 | N/A |
| Trimester | | |

As shown in Table No. 4, in trimester-specific logistic regression models controlling for chronic hypertension, diabetes, history of preeclampsia, twin gestation, and body mass index, copeptin concentration remained significantly associated with the development of preeclampsia.

The results indicate that testing characteristics are improved for women with no history of preeclampsia in the first and second trimester in comparison to the parent cohort of all comers.

### Discussion:

In the parent cohort and nested case control cohort, elevated copeptin is highly predictive in pregnant women for the development of preeclampsia when controlling for other covariates. However, surprisingly and unexpectedly, testing characteristics of copeptin for predicting preeclampsia actually improve in a nested case control study of women with no history of preeclampsia compared to women with a history of preeclampsia.

These results are particularly important because while prenatal care providers are likely to be watchful for signs and symptoms of preeclampsia in women with a history of the disease, they are also likely to be less vigilant in watching for signs and symptoms of preeclampsia in women with no history of the disease. Therefore, clinical testing for copeptin levels to detect preeclampsia in early pregnancy offers a further benefit of being more sensitive for predicting preeclampsia in a patient population with no history of the disease than in a population with a history of preeclampsia. Such testing is highly clinically relevant in that it allows identification of pregnant women with no history of preeclampsia that will develop preeclampsia and require closer monitoring or hospital care at an earlier stage in their pregnancy than was previously possible. Earlier identification of women who will develop preeclampsia enables earlier therapeutic intervention, which may help alleviate severity of the disease.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention. Percentages disclosed herein may otherwise vary in amount by ±10, 20, or 30% from values disclosed herein.

We claim:

1. A method of detecting copeptin in a patient, the method comprising:
obtaining a bodily sample from a patient;
applying the bodily sample to an assay adapted to detect copeptin in the bodily sample; and
detecting a level of copeptin in the bodily sample,
wherein the patient is a pregnant woman with no history of preeclampsia.

2. The method of claim 1, wherein the bodily sample comprises whole blood, serum, plasma, urine, tissue, cells, sweat, or tears.

3. The method of claim 2, wherein the bodily sample is a fresh sample or a frozen sample.

4. The method of claim 1, wherein the step of applying the bodily sample to the assay comprises:
a) combining the bodily sample with an assay solution comprising a copeptin-specific binding agent to form an assay mixture comprising copeptin bound with the copeptin-specific binding agent and applying the assay mixture to a substrate;
b) applying the bodily sample to a substrate and applying an assay solution to the substrate comprising a copeptin-specific binding agent to form an assay mixture; or
c) applying the bodily sample to a copeptin-specific binding agent bound to a substrate.

5. The method of claim 1, wherein the copeptin-specific binding agent comprises an antibody or an antibody fragment.

6. The method of claim 5, wherein the antibody or antibody fragment is linked to at least one of an enzyme, a nucleic acid tag, a prosthetic group, a fluorescent material, a luminescent material, a bioluminescent material, a radioactive material, a positron emitting metal, and a nonradioactive paramagnetic metal ion.

7. The method of claim 5, wherein the antibody is an IgG₁, IgG₂, IgG₃, IgG₄, IgA₁, IgA₂, IgM, IgE, or IgD antibody, and
wherein the antibody fragment is a Fab, a F(ab′)₂, a monospecific Fab₂, a bispecific Fab₂, a trispecific Fab₃, a monovalent IgG, an scFv, a bispecific diabody, a trispecific triabody, an scFv-sc, a minibody, an IgNAR, a V-NAR, an hcIgG, or a VhH.

8. The method of claim 1, wherein the bodily sample is obtained during the first trimester of pregnancy.

9. A method for identifying a treatment modality for a pregnant subject, the method comprising:
a) obtaining a bodily sample from the subject, wherein the sample is taken during the first trimester of the pregnancy;
b) measuring copeptin levels in the bodily sample using an assay; and
c) identifying a treatment modality for the pregnant subject based on an increased level of copeptin in the bodily sample as measured by the assay,
wherein the assay provides greater sensitivity for predicting the occurrence of preeclampsia in pregnant women with no history of the disease than in pregnant women with a history of the disease.

10. The method of claim 9, wherein the increased level of copeptin is predictive of the pregnant subject developing preeclampsia.

11. The method of claim 10, wherein the treatment modality is for the treatment of preeclampsia.

12. The method of claim 10, wherein the treatment modality comprises administration of at least one of an antihypertensive, a corticosteroid, an anticonvulsant, and a vaptan.

13. The method of claim 10, wherein the method enables an earlier initiation of treatment of preeclampsia during the pregnancy of the pregnant subject.

14. A kit for predicting the occurrence of preeclampsia in a subject, comprising:
a binding agent adapted to bind copeptin in a bodily sample taken from the subject during the first trimester of pregnancy, and
wherein the kit provides greater sensitivity for predicting the occurrence of preeclampsia in pregnant women with no history of the disease than in pregnant women with a history of the disease.

15. The kit of claim 14, wherein the binding agent comprises an antibody or an antibody fragment.

16. The kit of claim 15, wherein the antibody or antibody fragment is linked to at least one of an enzyme, a nucleic acid tag, a prosthetic group, a fluorescent material, a luminescent material, a bioluminescent material, a radioactive material, a positron emitting metal, a nonradioactive paramagnetic metal ion.

17. The kit of claim 15, wherein the antibody is an IgG₁, IgG₂, IgG₃, IgG₄, IgA₁, IgA₂, IgM, IgE, or IgD antibody, and
wherein the antibody fragment is a Fab, a F(ab′)₂, a monospecific Fab₂, a bispecific Fab₂, a trispecific Fab₃, a monovalent IgG, an scFv, a bispecific diabody, a trispecific triabody, an scFv-sc, a minibody, an IgNAR, a V-NAR, an hcIgG, or a VhH.

18. The kit of claim 14, wherein the kit enables quantification of copeptin in the bodily sample by quantitative PCR, epitope pull down via antibody-linked magnetic particles, column chromatography, gas chromatography, mass spectrometry, fluorescence, color change, flow cytometry, tissue staining, densitometry, western blot, or bio-barcode.

19. The kit of claim 18, wherein the kit further includes one or more binding agents adapted to bind one or more of cell-free fetal DNA, cell-free total DNA, and pregnancy-associated plasma protein A.

20. The kit of claim 14, wherein the kit further comprises at least one binding agent adapted to detect aneuploidy, alpha-fetoprotein, or human chorionic gonadotropin.
